(19) United States
(12) Patent Application Publication
(10) Pub. No.: US 2025/0177430 A1
(43) Pub. Date: Jun. 5, 2025

(54) Compositions Comprising NAD+ and Associated Methods of Treatment

(71) Applicant: David Henry Workman, Riverton, UT (US)

(72) Inventor: David Henry Workman, Riverton, UT (US)

(21) Appl. No.: 18/837,539

(22) PCT Filed: Feb. 10, 2023
(86) PCT No.: PCT/US2023/062354

§ 371 Date: Aug. 9, 2024

Related U.S. Application Data
(60) Provisional application No. 63/308,736, filed on Feb. 10, 2022.

(51) Int. Cl. A61K 31/7084; A61K 31/135; A61K 31/167; A61K 31/573; A61P 25/22; A61P 25/24; A61P 25/32

(52) U.S. Cl. CPC A61K 31/7084; A61K 31/135; A61K 31/167; A61K 31/573; A61P 25/22; A61P 25/24; A61P 25/32

(57) ABSTRACT

A composition is disclosed. The composition comprises an anesthetic and NAD+. In some embodiments, the anesthetic is lidocaine. In some embodiments, the composition comprises about 5 mg/ml lidocaine to about 20 mg/ml lidocaine. In some embodiments, the composition comprises about 20 mg/ml lidocaine. In some embodiments, the composition comprises about 50 mg/ml to about 500 mg/ml NAD+. In some embodiments, the composition comprises about 200 mg/ml NAD+. In some embodiments the combination comprises an anesthetic, NAD+, and an NMDA receptor antagonist. In some embodiments, the NMDA receptor antagonist is ketamine. In some embodiments, the composition comprises about 10 mg/ml ketamine to about 200 mg/ml ketamine. In some embodiments, the composition comprises about 50 mg/ml ketamine, In some embodiments, the combination comprises an anesthetic, NAD+, an NMDA receptor antagonist, and a corticosteroid. In some embodiments, the corticosteroid is dexamethasone. In some embodiments, the composition comprises about 0.5 mg/ml dexamethasone to about 3 mg/ml dexamethasone.

### CROSS REFERENCE TO RELATED APPLICATIONS

This application is a national phase entry under 35 U.S.C. § 371 of International Patent Application PCT/US2023/062354, filed Feb. 10, 2023, designating the United States of America and published in English as International Patent Publication WO 2023/154844 on Aug. 17, 2023, which claims the benefit of U.S. Provisional Patent Application Ser. No. 63/308,736, filed Feb. 10, 2022, the entireties of which are hereby incorporated by reference.

### TECHNICAL FIELD

This invention relates to compositions comprising NAD+ and their use for the treatment of chemical dependency including withdrawal symptoms, withdrawal cravings, and in treating anxiety and depression.

### BACKGROUND

NAD+ has been known since the 1960s to be very effective in the treatment of withdrawal symptoms as well as cravings in the settings of opiate and alcohol abuse. NAD+ has primarily been administered intravenously.

### SUMMARY

In a first aspect, the disclosure provides a composition comprising an anesthetic and NAD+. In some embodiments, the concentration of anesthetic in the compositions is sufficient to produce anesthesia in a tissue on which the composition is placed in contact with. The anesthetic in the composition is selected from the group consisting of lidocaine, prilocaine, cocaine, sameridine bupivacaine, articaine, proparacaine, procaine, and amylocaine. In some embodiments, the anesthetic is lidocaine. In some embodiments, the composition comprises about 3 mg/ml lidocaine to about 20 mg/ml lidocaine. In some embodiments, the composition comprises about 20 mg/ml lidocaine. In some embodiments, the composition comprises about 25 mg/ml to about 500 mg/ml NAD+. In some embodiments, the composition comprises about 200 mg/ml NAD+.

In a second aspect, the disclosure provides a composition comprising an anesthetic, NAD+, and an NMDA receptor antagonist. The anesthetic in the composition is selected from the group consisting of lidocaine, prilocaine, cocaine, sameridine bupivacaine, articaine, proparacaine, procaine, and amylocaine. The NMDA receptor antagonist in the composition is selected from the group consisting of pethidine, levorphanol, ketamine, dextromethorphan (DXM), methoxetamine (MXE), and ketobemidone. In some embodiments, the NMDA receptor antagonist in the composition comprises ketamine. In some embodiments, the concentration of ketamine in the composition comprises about 10 mg/ml ketamine to about 200 mg/ml ketamine. In some embodiments, the concentration of ketamine in the composition comprises about 50 mg/ml ketamine. In some embodiments, the concentration of ketamine in the composition comprises about 100 mg/ml ketamine.

In a third aspect, the disclosure provides a composition comprising an anesthetic, NAD+, an NMDA receptor antagonist, and a corticosteroid. The corticosteroid is selected from the group consisting of cortisone, prednisone, prednisolone, methylprednisolone, dexamethasone, betamethasone, and hydrocortisone. In some embodiments, the corticosteroid is dexamethasone. In some embodiments, the concentration of dexamethasone in the composition comprises about 0.5 mg/ml dexamethasone to about 10 mg/ml dexamethasone.

In a fourth aspect the disclosure provides a method for the treatment of anxiety, depression, or chemical dependency in a subject in need thereof, the method comprising: administering to the subject, any of the compositions described above. In some embodiments, the subject is administered at least 1ml of the composition. In some embodiments, the composition is administered to the subject daily for five days. In some embodiments, the composition is administered at two-month to six-month intervals. In some embodiments, the chemical dependency is opioid dependency or alcohol dependency.

In a fifth aspect the disclosure provides a method for the treatment of anxiety, depression, or chemical dependency. In some embodiments, the chemical dependency is opioid dependency or alcohol dependency.

In a sixth aspect the disclosure provides a method for the use of NAD+ and an anesthetic in the manufacture of a treatment for anxiety, depression, or chemical dependency. In some embodiments, the use of NAD+ and an anesthetic in the manufacture of a treatment for anxiety, depression, or chemical dependency, wherein the chemical dependency is opioid dependency or alcohol dependency.

Further aspects and embodiments are provided in the foregoing drawings, detailed description and claims.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “pterygopalatine fossa” is an inverted pyramid-shaped space in the cranium. The boundaries of the pterygopalatine fossa are the junction of the maxilla, palatine, and sphenoid bones. The contents of the pterygopalatine fossa include fat, the pterygopalatine or sphenopalatine ganglion (PPG), the maxillary division (V2) of the trigeminal nerve and its branches, which include the zygomatic nerve, the posterior superior alveolar nerve(s), and the infraorbital nerve (ION), the Vidian (pterygoid) nerve, the distal branches of the maxillary artery, and a few emissary veins. This fossa has 7 ingress and egress tracts which have earned it the nickname “the neurovascular crossroads of the head”.

As used herein, “pterygopalatine or sphenopalatine ganglion” is meant to refer to, one of the largest clusters of nerve bodies outside of the central nervous system. The sphenopalatine ganglion is responsible for much of the autonomic regulation inside the cranium and has pass-through pain fibers from the meninges and the trigeminal system.

As used herein, “local anesthetic” is meant to refer to agents that affect the nerve impulses in tissue, typically by blocking the sodium channels facilitating these nerve impulses. This results in the reversible and temporary loss of sensation in a defined area of the body. Local anesthetics include, but are not limited to, lidocaine, prilocaine, cocaine, sameridine bupivacaine, articaine, proparacaine, procaine, amylocaine.

As used herein, “nerve block” is meant to refer to local anesthetic agents being administered adjacent to nerves, typically large nerves, and causing loss of sensation to the larger area supplied by those nerves.

As used herein, “NMDA antagonist” is meant to refer to a class of drugs that work to antagonize or inhibit the action of the N-methyl-D-aspartate receptor (NMDAR). These drugs are commonly used for anesthesia in animals and humans. NMDA receptor antagonists often induce a state known as dissociative anesthesia. NMDA receptor antagonists include, but are not limited to, pethidine, levorphanol, ketamine, dextromethorphan (DXM), methoxetamine (MXE), and ketobemidone.

As used herein, “sphenopalatine ganglion block device” is meant to refer to specialized catheters which are inserted along the anterior nasal passage and allow the administration of substances to the mucosal membranes covering the sphenopalatine foramen. The catheters are comprised of a flexible sheath with an inner, extendible catheter with a curved tip. The opening of the catheter tip is designed to direct anesthetic in a superior, lateral, and posterior direction. There are several sphenopalatine ganglion block devices commercially available, including SphenoCath, Allevio SPG Nerve Block Catheter, and Tx360 Nasal Injector.

As used herein “corticosteroid” is meant to refer to substances produced that closely resemble cortisol, which is produced in the adrenal gland. Corticosteroids are often used to reduce inflammation. Corticosteroids include but are not limited to cortisone, prednisone, prednisolone, methylprednisolone, dexamethasone, betamethasone, and hydrocortisone.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those in the art. For example, in the appended claims, any of the claimed embodiments can be used in any combination.

The pterygopalatine fossa has been an area of interest for over 100 years. One of the reasons for interest in the pterygopalatine fossa is the ability to treat patients by administering treatment into this fossa. The treatments will be received by the nerves and the ganglion in the fossa.

In modern times this target was first approached using a curved needle through the roof of the mouth in 1906. This technique has been replaced by the infra zygomatic needle approach for direct access, which requires anesthesia and fluoroscopy, and is not without risk.

Over the past several decades most practitioners have approached this target through the nasal passage. There are two small holes or foramen in the bone in the superior/posterior aspect of the nasal cavity near the sphenoethmoid recess. These holes, known as the sphenopalatine foramen, are covered by a 2-3 mm membrane which allows for fairly rapid absorption of medications or substances delivered to this target into the pterygopalatine fossa and hence, to the sphenopalatine ganglion and through the egress tracts into the central nervous system.

Until recently the most popular, and probably the most effective way of delivering medications to this target through the nasal cavity was by use of long cotton-tipped applicators soaked in medication and then placed up into the most superior and posterior aspects of the nasal cavity on both sides and left in place for several minutes. This is a somewhat difficult technique to use without causing nosebleed, and it is difficult to regulate the amount of medication actually delivered to the target.

The SphenoCath, an FDA cleared intranasal medication delivery device, was introduced to the market. The SphenoCath utilizes a specialized curved flexible catheter attached to a syringe. The syringe enables a determined amount of substance to be measured and administered. This allowed for very reliable delivery of a determined amount of medication to this target area in the superior/posterior aspect of the nasal cavity much more quickly and with much less chance of trauma or discomfort.

The inventor has treated many migraine and facial pain patients over the past eight or nine years using this catheter to deliver an anesthetic such as lidocaine. This procedure, usually referred to as a sphenopalatine ganglion block, is now talked about and presented in most headache conferences in the United States. More generally, administering an anesthetic such as lidocaine to a nerve is referred to as a nerve block. Additionally, the inventor has trained many doctors and other practitioners around the country in this sphenopalatine ganglion block technique.

Due in part to the success of the sphenopalatine ganglion block technique the inventor believed that this technique could be adapted for use as a treatment for other indications, utilizing other medications and substances. Among those other indications utilizing other medications the inventor discussed the use of NAD+. It was thought that NAD+ could be effective in the treatment of mood disorders such as anxiety and depression.

NAD+ has been used in many other forms including intravenous infusion, intranasal sprays, sublingual lozenges, intramuscular or subcutaneous injection, creams and patches. It is not effective to take it orally. Traditional nasal sprays do not target or significantly access the sphenopalatine foramen. They rely on mucosal absorption in the anterior portion of the nasal cavity and the turbinates.

The inventor made a first attempt at treating acute chemical dependency, specifically opiate withdrawal using the SphenoCath technique to deliver lidocaine alone. Inventor's believe was that since a significant number of the chemical dependency, such as withdrawal symptoms experienced by people are a result of dysautonomia, and that this would be a safe and simple thing to try. Other attempts have been made utilizing an anesthetic in combination with NAD+, and further treatments have been performed using a composition that combines an anesthetic such as lidocaine, NAD+, and an NMDA receptor antagonist such as ketamine.

The inventor has discovered that there is a very synergistic effect when lidocaine, NAD+, and ketamine are administered together. As previously described, the inventor has used lidocaine with this delivery method for several years for the treatment of chronic migraine, acute migraine, cluster headaches, trigeminal neuralgia, postherpetic neuralgia, post-concussion headaches, and a regional pain syndrome of the jaw case. Treatment with lidocaine through the sphenopalatine foramen into the pterygopalatine fossa for these conditions has been successful in most cases.

The addition of NAD+ has significantly enhanced the effect on chemical dependency, treating both withdrawal symptoms and cravings. It has its own effect on mood disorders as well. However, the addition of ketamine has proven extremely effective as also described. In comparing the treatment treating patients who have experienced the mixture of NAD+, lidocaine, and ketamine with just ketamine, the experience of using just ketamine is dramatically different and not in a positive way. The IV and intranasal ketamine alone experience is typically a more intense and dissociative experience.

A patient may receive medication through the pterygopalatine fossa in a number of ways. A curved needle may be inserted into this location through the roof of the mouth. This method is effective at administering a determined amount of medication; however, this technique is rarely used at the present time. Another technique is infra zygomatic needle approach. A needle is inserted under the zygomatic process (commonly referred to as the cheekbone). This process requires anesthesia and fluoroscopy, which means a patient must be sedated and under an x-ray so that the doctor can see where the medication is being injected.

An alternative method for administering medication to the pterygopalatine fossa through the sphenopalatine foramen is to soak absorbent applicators in medication and place them in position within the nasal cavity. This technique does not require the use of radiological imaging equipment such as x-rays, however nosebleeds and discomfort are frequent side effects of this technique. Another disadvantage to utilizing absorbent applicators is the inability to precisely monitor or regulate the amount of medication administered in this way.

A device called the SphenoCath has been used to administer medication to the sphenopalatine foramen. The SphenoCath is a curved flexible catheter with a semi-rigid retractable sheath overlaying the catheter attached to a syringe. The SphenoCath is inserted through the nostril, until the tip of the SphenoCath reaches the anterior roof of the nasal cavity. The sheath of the SphenoCath is retracted allowing the curved inner catheter to drop over the anterior aspect of the middle turbinate. With the head correctly positioned medication is reliably dispensed to the area of the membrane covering the sphenopalatine foramen, and then through absorption into the pterygopalatine fossa and the nerves contained therein.

Administering medication to and through the pterygopalatine fossa has been shown to be effective for several conditions, including acute migraine headaches, cluster headache, trigeminal neuralgia, withdrawal symptoms, anxiety, depression, and others. The condition experienced by the patient generally determines the combination and dose of the medication administered.

Migraine and other headache conditions are generally treated with an anesthetic such as lidocaine. In some embodiments, the anesthetic is administered through a single nostril, to a single sphenopalatine fossa. In other embodiments, the anesthetic is administered through both nostrils, to both sphenopalatine fossa. In some embodiments, 3 mg/ml to 30 mg/ml local anesthetic is administered. In further embodiments, 5 mg/ml to 20 mg/ml of local anesthetic is administered. In yet other embodiments, the amount of local anesthetic administer is ≤3, ≤4, ≤5, ≤6, ≤7, ≤8, ≤9, ≤10, ≤11, ≤12, ≤13, ≤14, ≤15, ≤16, ≤17, ≤18, ≤19, ≤20 mg/ml of local anesthetic is administered. In yet other embodiments, the amount of local anesthetic administer is ≤5, ≤6, ≤7, ≤8, ≤9, ≤10, ≤11, ≤12, ≤13, ≤14, ≤15, ≤16, ≤17, ≤18, ≤19, ≤20 mg/ml of lidocaine is administered.

Treating chemical dependency, including withdrawal symptoms is in some embodiments accomplished with a composition of an anesthetic and NAD+. In some embodiments, the local anesthetic is utilized in the same amounts as utilized in treating headache related conditions. Combining NAD+ with a local anesthetic, the NAD+ is administered in amounts of between 25 mg/ml to 250 mg/ml. In other embodiments, the amount of NAD+ is between 50 mg/ml and 400 mg/ml. In further embodiments, the amount of NAD+ is 75 mg/ml and 150 mg/ml. In yet other embodiments, the amount of NAD+ is 100 mg/ml. In other embodiments the concentration of NAD+ is ≤50, ≤75, ≤100, ≤120, ≤140, ≤150, ≤160, ≤170, ≤180, ≤190, ≤200, ≤220, ≤240, ≤260, ≤280, ≤300, ≤320, ≤340, ≤360, ≤380, ≤400, ≤420, and ≤440.

The composition of lidocaine and NAD+ is administered through the nasal passage, once the composition has been placed on the mucus membrane, the patient is encouraged to remain in place to allow the composition to pass through the membrane. In some embodiments, the patient rests for between 5 and 25 minutes. In other embodiments, the patient rests for between 10 and 20 minutes. In yet other embodiments, the patient rests for 15 minutes. In some embodiments, the neck is extended about 30 degrees. In some embodiments, the patient rotates the head approximately 30 degrees to either side, then approximately 30 degrees to the other side, and finally leaves the head in neutral position. In these embodiments the first rotation is between 5 and 15 minutes, the second rotation is between 5 and 15 minutes, and the neutral position is between 5 and 15 minutes. In some embodiments, the head is rotated every 5 minutes, for 20 minute to 30-minute sessions. In some embodiments, the neutral position is not used as a position for the head.

Practitioners generally express treatment in terms of amount of a substance administered. For example, a practitioner may administer 200 mg of a medication. The medication is available in 200 mg/ml concentrations in a liquid therefore a dose of 200 mg would be 1 ml of the medication. If the practitioner desired to administer 400 mg of the medication, they would administer 2 ml of available medication.

When utilizing medications in combination the practitioner will likely have more than one medication in solution and the concentration of each medication in the solution will depend on the overall volume of the combination. For example, one medication may be available in 200 mg/ml concentrations and another in 100 mg/ml concentrations. The practitioner wishes to administer 300 mg of the first medication and 50 mg of the second medication. The combination will then include 1.5 ml of the first medication and 0.5 ml of the second medication for a total volume of 2 ml. The resulting concentration of the first medication will be 150 mg/ml and 25 mg/ml of the second medication.

The composition of lidocaine and NAD+administered by a device such as the SphenoCath needs to be administered as a liquid, which can be either aqueous or viscous. The combinations of the medications of the composition are dissolved in a liquid. The SphenoCath has a capacity to deliver as much liquid as can be held in a syringe. The finite volume of the nasal passage and the rate of diffusion through the membrane covering the sphenopalatine foramen limits the volume of liquid that can be administered. Generally, the volume of liquid administered is between 0.3 ml and 5 ml. In some instances, as much as 6 ml of liquid could be administered in the nasal cavity. Larger volumes become increasingly difficult to administer as any patient must wait for the liquid to diffuse across the mucosal membrane in the nasal cavity. The larger the volume the longer the patient must wait.

In other embodiments, chemical dependency is treated, with a composition of an anesthetic, NAD+, and an NMDA receptor antagonist. In embodiments, utilizing a local anesthetic, NAD+, and an NMDA receptor antagonist, the local anesthetic and the NAD+ are administered through the SphenoCath in volumes from 1 ml to 4 ml in the concentrations described previously. An NMDA receptor antagonist is added in some embodiments, in concentrations between 5 mg/ml and 150 mg/ml. In other embodiments, the NMDA receptor antagonist is added in concentrations between 10 mg/ml and 40 mg/ml. In yet other embodiments, the NMDA receptor antagonist is added in concentrations between 15 mg/ml and 30 mg/ml. In further embodiments, the NMDA receptor antagonist is ketamine. In some embodiments, where the composition includes ketamine the concentration of ketamine is 12.5 mg/ml. In other embodiments the concentration of ketamine is ≤5, ≤10, ≤20, ≤30, ≤40, ≤50, ≤60, ≤70, ≤80, ≤90, ≤100, ≤110, ≤120, ≤130, ≤140, ≤150, and ≤160.

In some embodiments, chemical dependency is treated with the combination of a local anesthetic, NAD+, and an NMDA antagonist with the addition of a corticosteroid. Examples of corticosteroid used in the combination include but are not limited to cortisone, prednisone, prednisolone, methylprednisolone, dexamethasone, betamethasone, and hydrocortisone. The corticosteroid is added to the combination. In some embodiments, 1 ml to 4 ml of the combination is administered, and the concentration of the corticosteroid in the combination is between 0.5 mg/ml to 10 mg/ml.

In some embodiments, anxiety is treated utilizing a composition of a local anesthetic and NAD+. In some embodiments, 1 ml to 4 ml of the combination of local anesthetic and NAD+ is administered. The local anesthetic is prepared in concentrations of 5 mg/ml to 20 mg/m, and the NAD+ is prepared in concentration of between 50 mg/ml to 500 mg/ml. In further embodiments, 0.5 ml to 4 ml of a combination comprising; 10 mg/ml to 20 mg/ml of local anesthetic and between 50 mg/ml and 500 mg/ml NAD+. In yet other embodiments, 0.5 ml to 4 ml of the combination comprising 20 mg/ml of local anesthetic and a concentration of 200 mg/ml NAD+ is administered.

In some embodiments, anxiety is treated utilizing a composition of a local anesthetic, NAD+, and an NMDA receptor antagonist. In some embodiments, 1 ml to 4 ml of a combination of a local anesthetic at a concentration of 5 mg/ml to 20 mg/ml, NAD+ at a concentration of between 50 mg/ml to 500 mg/ml, and an NMDA receptor antagonist at a concentration of between 5 mg/ml and 200 mg/ml is administered. In further embodiments, the combination comprises 10 mg/ml to 20 mg/ml of local anesthetic, between 100 mg/ml and 400 mg/ml NAD+, and between 10 mg/ml and 150 mg/ml of an NMDA receptor antagonist is administered. In yet other embodiments, 1 ml to 4 ml of a combination of a local anesthetic at a concentration of 20 mg/ml. NAD+ at a concentration of 200 mg/ml, and an NMDA receptor antagonist at a concentration of between 60 mg/ml and 125 mg/ml. In some embodiments, 1 ml to 4 ml of a composition comprising 20 mg/ml lidocaine, 200 mg/ml NAD+, and 40 mg/ml ketamine is administered.

In some embodiments, depression is treated utilizing a composition of a local anesthetic and NAD+. In some embodiments, 1 ml to 4 ml of a combination, where the concentration of local anesthetic is from 5 mg/ml to 40 mg/ml and the concentration of NAD+ is from 50 mg/ml to 500 mg/ml. In further embodiments, 0.5 ml to 4 ml of a combination where the concentration of the local anesthetic is between 10 mg/ml and 40 mg/ml, and the concentration of NAD+ is between 100 mg/ml and 400 mg/ml, is administered. In yet other embodiments, 1 ml to 4 ml of a combination where the concentration of the local anesthetic is between 20 mg/ml and the concentration of NAD+ is 200 mg/ml, is administered.

In some embodiments, depression is treated utilizing a composition of a local anesthetic, NAD+, and an NMDA receptor antagonist. In some embodiments, between 1 ml and 4 ml where the concentration of the local anesthetic is from 5 mg/ml to 20 mg/ml, the concentration of NAD+ is from 50 mg/ml to 500 mg/ml, and the concentration of the NMDA receptor antagonist is from 10 mg/ml to 100 mg/ml, is administered. In further embodiments, between 1 ml and 4 ml of the combination is administered where the concentration of the local anesthetic is between 10 mg/ml and 20 mg/ml, the concentration of NAD+ is between 100 mg/ml and 400 mg/ml, and the concentration of NMDA receptor antagonist is between 20 mg/ml and 200 mg/ml. In yet other embodiments, between 1 ml and 4 ml of the combination is administered where the concentration of the local anesthetic is between 20 mg/ml, the concentration of NAD+ is 200 mg/ml, the concentration of NMDA receptor antagonist is between 30 mg/ml and 200 mg/ml. In some embodiments the composition is 20 mg/ml lidocaine, 200 mg/ml NAD+, and 50 mg/ml ketamine.

The individual components of the composition can be administered individually. As has been explained, the administration of an anesthetic such as lidocaine has proved useful in the treatment of headache related condition, including migraine headaches. Administering NAD+ individually, can treat chemical dependency, anxiety, and depression, however the patient experience upon receiving NAD+ individually is less pleasant and can lead to discomfort for the patient. Pairing NAD+ with an anesthetic such as lidocaine has a synergistic effect in that the components work together and there is little to no discomfort for the patient. Similarly, an NMDA receptor antagonist such as ketamine may be administered individually, however the dissociative nature of an NMDA receptor antagonist such as ketamine is more pronounced when administered individually and can lead to discomfort for the patient. Adding an NMDA receptor antagonist such as ketamine to the composition of an anesthetic such as lidocaine and NAD+ has further synergistic effects. The NAD+ significantly diminishes the dissociative effects of the NMDA receptor antagonist.

It is not always possible or practical to administer the composition through the pterygopalatine fossa. There are many reasons why administration through the pterygopalatine fossa is not possible including obstruction of the nostrils, the inability of a patient to remain still, and other difficulties. For this reason other administration pathways are available. Other administration methods and locations will have other benefits including specific location treatment, case of access to treatment location, case of method of administration, and other benefits. One advantage to administration through the pterygopalatine fossa is the access to the nerves and nerve ganglion, other methods and locations that have ready access to nerves are also good places of administration of the combination. Such methods include epidural administration and intrathecal administration. Locations and methods near the brain are also good locations because of access to nerves and to the brain, such methods include intracerebroventricular administration, intracochlear, intranasal, transnasal, inhaled, and intratympanic. Administration to the bloodstream gives widespread distribution so intravenous and intraarterial administration can be used when such distribution is desired. Some membranes are more permeable and more likely to facilitate the administration of the combination when administered thereto, such membranes and administration methods include sublingual, buccal, rectal, and vaginal. Administration into a tissue provides targeted results, such methods include intraosseous, intramuscular, transdermal, and subcutaneous. For a general distribution oral and enteral administration are standard methods of administration. Epicutaneous administration will generally have a localized affect near the administration site. Each of these methods is a path for administration of the combination.

### EXAMPLES

A dosing protocol was developed over the course of treating patients.

Lidocaine is available in concentrations of
1% (10 mg/ml),
2% (20 mg/ml), and
4% (40 mg/ml).

NAD+ is available as a solid and is typically made in concentrations of
50 mg/ml and
100 mg/ml.

Early on the lidocaine and the NAD+ were mixed separately and then added to the SphenoCath individually. In a later iteration the lidocaine was added to the NAD+. Ideally the volume infused should be 3 ml or less.

The combined NAD+/Lidocaine concentrations were
100 mg/ml NAD+ 5 mg/ml Lidocaine,
200 mg/ml NAD+ 5 mg/ml Lidocaine,
100 mg/ml NAD+ 20 mg/ml Lidocaine, and
200 mg/ml NAD+ 20 mg/ml Lidocaine.

The ketamine was prepared in concentrations of
50 mg/ml and
100 mg/ml.

Inventor treated patients according to overall amount of each medication. Therefore, if the inventor administered a dose of 40 mg lidocaine, the dose would be 4 ml of 1% (10 mg/ml) lidocaine, 2 ml of 2% (20 mg/ml) lidocaine, and 1 ml of 4% (40 mg/ml) lidocaine. If the inventor wished to administer a dose of 150 mg of NAD+, the dose would be 3 ml of 50 mg/ml NAD+, or 1.5 ml of 100 mg/ml NAD+. When NAD+ and lidocaine were both administered, for example 40 mg lidocaine and 150 mg NAD+. When using 2% lidocaine and 100 mg/ml NAD+ the total final volume would be 2 ml lidocaine plus 1.5 ml NAD+ totaling 3.5 ml, resulting in a final concentration of lidocaine of 11.4 mg/ml and a final concentration of NAD+ of 43 mg/ml. Typically a practitioner wishing to treat patients will use the dosing protocol developed by the inventor.

### Example 1

A significant portion of the symptoms of chemical dependency including the symptoms of opiate withdrawal are manifestations of autonomic chaos in the central nervous system. Patient was a 52-year-old male experiencing chemical dependency, specifically opiate withdrawal symptoms, patient was treated, using the SphenoCath and lidocaine, twice 48 hours apart. Patient felt that symptoms were noticeably improved. He had experienced opiate withdrawal in the past, so was familiar with the symptoms of withdrawal. Patient 1 was able to go through withdrawal without the use bupenophrine, which was necessary for previous detox episodes.

### Example 2

As NAD+ has been used intravenously, intramuscularly, in topical preparations, in intranasal sprays, sublingual lozenges, and other treatments. Inventor determined to utilize the SphenoCath to deliver NAD+ to the sphenopalatine foramen. Patient was a 57-year-old married mother of five who had battled severe anxiety and depression for many years. Patient had been successfully treated for migraines for five to six years using lidocaine with this catheter and delivery method. Patient's symptoms had gone from having some form of headache nearly every day and being bedbound for several days a month with migraines to having very rare migraines and almost never any bedbound days.

Patient suffered from anxiety for Patient's entire life. A particularly troubling event augmented the anxiety, particularly in relation to flying. Approximately, 20 years ago Patient was preparing to fly to California. Despite having flown to Europe and both US coasts previously, for some reason, when patient saw the doors of the airplane closed patient started into a panic attack which escalated to the point that the airplane had to be turned around from the runaway and taken back to the terminal where patient was taken off of the plane to the cheers of some of the passengers. Since that day the thought of flying and even sometimes seeing an airliner fly overhead caused tremendous anxiety. Patient had been treated with traditional antidepressants and antianxiety agents for many years and had received excellent counseling all to some effect, but never resulting in patient being able to fly.

Patient was started on a treatment regime of NAD+ and lidocaine. After six treatments of NAD+ and lidocaine over the course of three weeks, along with the continuation of patient's other medications and tools, patient was able to very successfully handle a turbulent three-hour airline flight and the return. Patient admitted to having felt some anxiety upon entry into the airport parking lot, and a low-grade curiosity about whether anxiety or panic would again set in, but otherwise tolerated the experience very well and has been on other airline flights since. Patient continued treatment over the course of a six-month period on an as-needed basis, which ranged from twice a week to every other month.

### Example 3

The patient from the previous example also suffered from refractory depression. Ketamine has received renewed interest for treatment of such depression. Patient 2 had discussed trying IV ketamine; however, it was never attempted. Inventor researched the use of Esketamine and knew of physicians prescribing compounded ketamine nasal spray, and due to the success of the NAD+ treatment for anxiety, inventor discussed with patient the idea of trying the addition of ketamine to the NAD+/lidocaine blend which had been successfully utilized. Treatment began with the addition of a very low 25 mg dose of ketamine to the NAD+/lidocaine treatment. Inventor and patient decided to begin with the fairly typical six treatment induction phase used by IV ketamine clinics, but deliver it using the SphenoCath to the sphenopalatine foramen.

The patient reported that after the second treatment “obsessive thoughts just stopped, like a train hitting a wall”. Patient 2 added “I could not make myself obsess about anything”. Patient 2 further described feeling “happy” and “laughing”, which had not been a common experience lately for patient. Patient 2 further described feeling “floaty” and “tingly” and unable walk straight or drive for about an hour after the treatments. However, when treated with the lidocaine, NAD+, and ketamine composition, Patient 2 had no dissociative experience despite the fact that the dose was increased to 40 mg of ketamine over the course of the first 2 weeks. Treatment then went to an as-needed basis with this combination of NAD+, lidocaine, and ketamine. The treatment has turned into about once a week to once every 2 months for the past 18 months with very good results. Patient has been able to finally eliminate the use of Wellbutrin and Xanax.

Of note, patient did try IV ketamine at a ketamine clinic on one occasion about six months into the treatment regime. Patient had them shut it off after having received about 30 mg because it was a very dissociative and unpleasant experience for patient. After that single treatment was discontinued patient felt fine within 30-45 minutes. Patient further felt that the effect on mood was probably about the same as the intranasal treatments. It appears that the combination of NAD+ and lidocaine, with the ketamine, has a synergistic and buffering effect. This effect seems quite positive when delivered as a combination and to the location described.

### Example 4

A cohort comprised of a family, each of whom received treatment for various forms of anxiety and depression.

Patient 4 was a 64-year-old man and the father of the family. Patient suffered from anxiety. Due to a traumatic experience involving standing close to a TSA agent who was shot and killed along with another TSA agent in an airport in Los Angeles. Patient then had a gun held to his head and was asked if he was a TSA agent, to which Patient answered that patient was not. Patient 4 describes looking at the man holding the gun to patient's head and watching gunman decide whether to believe patient or not. Patient's wife was crouched under a podium in front of patient at the time.

Patient 4's anxiety made it very difficult to join his family in crowded venues and made it challenging for patient to perform duties such as conducting church meetings and speaking in front of large groups. Patient 4 had been receiving NAD+, lidocaine, and ketamine treatments every two to three months for three series of four to six treatments. Patient 4 had tolerated the treatment with very good results. Patient 4 agreed to receive the last treatment of the third series with just ketamine, at a slightly lower dose than the combined treatment received the day before. Patient 4 was very agitated and uncomfortable 15 minutes after the intranasal delivery to the sphenopalatine foramen. With some coaxing, Patient 4 was convinced to lay back down and the NAD+ and lidocaine components of the dose which had been included in the previous treatments, were administered. Patient 4 described “immediate calm” after receiving the NAD+. Patient 4 called the next day while driving to Colorado to express “feeling great”.

The initial series of six treatments once a day for six days occurred between December 2 and December 7 of 2020 with gradually increasing doses of a mixture of NAD+, ketamine, and lidocaine. The dose was initiated at low concentrations to which Patient 4 described very little effect on mentation after the first treatment. Treatment started with 1.8 ml at a concentration of 55 mg/ml of NAD+, 7 mg/ml Ketamine, and 3.3 mg/ml lidocaine. The second treatment was 2 ml of the combination with concentrations of 70 mg/ml NAD+, 9 mg/ml ketamine, and 5 mg/ml lidocaine. The third treatment was 2 ml of the combination with concentrations of 80 mg/ml NAD+, 11.25 mg/ml ketamine, and 5 mg/ml lidocaine. The fourth treatment was 2 ml of the combination with concentrations of 80 mg/ml NAD+, 12.5 mg/ml ketamine, and 5 mg/ml. The next three treatments were 2 ml of the combination with concentrations of 90 mg/ml NAD+, 15 mg/ml ketamine, and 5 mg/ml lidocaine. Then 2 ml of the combination with concentrations of 100 mg/ml NAD+, 18.75 mg/ml ketamine, and 5 mg/ml lidocaine. The dose was adapted to 2 ml of the combination with concentrations of 100 mg/ml NAD+, 21.25 mg/ml ketamine, and 20 mg/ml lidocaine. The next treatment was 2 ml of the combination with concentrations of 100 mg/ml NAD+, 20 mg/ml ketamine, and 5 mg/ml lidocaine. Several of the treatments included a rotation of the head. For example, the combination was administered to the Patient and the head was rotated 30 degrees to the right for 5 minutes, then 30 degrees to the left for 5 minutes and then neutral for 5 minutes. Other times the head was rotated to the right at 30 degrees for 5 minutes, then 30 degrees to the left for 5 minutes and then neutral for 5 minutes. Patient 4 was being treated along with other family members including a son and daughter, the treatments to the son and daughter will be described later. During the third treatment the family described that the evening before they had found themselves laughing and joking around in the hotel room in a way quite different from their previous family dynamics and personalities. Patient 4 was generally reticent to describe symptoms prior to treatment and self-scored quite low on the GAD 7 and PHQ 9 anxiety and depressions screening tools that each of these patients fill out prior to treatment and 1 month after. Patient 4's pre-and post-treatment scales were essentially the same. However, the family descriptions were more reveling including the wife's description which included the statement “he is a different man”.

Patient 5 is the eldest daughter, a 32-year-old mother of 2, who has battled depression and anxiety since about age 16, and has been on antidepressants since age 17, currently Wellbutrin and Viibryd for the past 3 years. Patient 5 flew from Denver for this series of treatments and underwent a similar six-day treatment regimen utilizing NAD+, lidocaine, and ketamine. Patient 5 experienced some nausea after the first treatment but not after subsequent treatments. As with her father, Patient 5, the dose of the NAD+ and ketamine was gradually increased, but the lidocaine was kept constant. The first treatment was with 1.8 ml at a concentration of 55 mg/ml of NAD+, 7 mg/ml Ketamine, and 3.3 mg/ml lidocaine. The second treatment was 2 ml of the combination with concentrations of 70 mg/ml NAD+, 18.75 mg/ml ketamine, 5 mg/ml lidocaine. Third treatment composition was 2 ml of the combination with concentrations of 50 mg/ml NAD+, 10 mg/ml, and 5 mg/ml lidocaine. Fourth and fifth treatment compositions were 2 ml of the combination with concentrations of 60 mg/ml NAD+, 10 mg/ml, and 5 mg/ml lidocaine. Patient 5 subjectively reported feeling much better emotionally at the end of the first week and in fact after the first two or three treatments. Patient 5 completed the GAD 7 and PHQ 9 screening tools before treatment and two weeks after completion of the series. Patient 2 GAD 7 scores went from 14 to 3 and PHQ 9 scores went from 13 to 2 in that three-week period. The effect of the treatment is felt early on and persists for several weeks. Patient returned for “booster” treatments four months later. The first “booster” treatment was 2 ml of the combination with concentrations of 60 mg/ml NAD+, 10 mg/ml ketamine, and 5 mg/ml lidocaine. The treatment regime also included 30-degree rotation to the right for 5 minutes, then 30-degree rotation to the left for 5 minutes, finally neutral for 5 minutes. Second booster dose was 2 ml of the combination with concentrations of 60 mg/ml NAD+, 12.5 mg/ml ketamine, and 5 mg/ml lidocaine. The third booster dose was 2 ml of the combination with concentrations of 60 mg/ml NAD+, 15 mg/ml ketamine, and 5 mg/ml lidocaine. The fourth booster dose was 2 ml of the combination with concentrations of 60 mg/ml NAD+, mg/ml ketamine, and 20 mg/ml lidocaine. Seven months later Patient 5 returned for a second round of booster treatments 0.5 ml of the combination with concentrations of 300 mg/ml NAD+, 100 mg/ml ketamine, and 20 mg/ml lidocaine. 0.5 ml of the combination with concentrations of 400 mg/ml NAD+, 130 mg/ml ketamine, and 20 mg/ml lidocaine. Dose was 0.5 ml of the combination with concentrations of 400 mg/ml NAD+, 150 mg/ml ketamine, and 20 mg/ml lidocaine.

Patient 6 was a 28-year-old man and son of Patient 4. Patient 6 has tried antidepressants in the past without success. Patient 6 attempted to treat mood issues primarily by spending two to three hours per day in the weight room. Patient 6 received similar treatments of gradually increasing regimen of NAD+, lidocaine and ketamine through the SphenoCath. The first treatment was 1.8 ml of the combination with concentrations of 55 mg/ml NAD+, 14 mg/ml ketamine, 3 mg/ml. Second treatment was 2.6 ml of the combination with concentrations of 54 mg/ml NAD+, 13 mg/ml ketamine, 4 mg/ml. Third treatment of 3 ml of the combination with concentrations of 53 mg/ml NAD+, 15 mg/ml ketamine, 7 mg/ml lidocaine. Fourth treatment of 3 ml of the combination with concentrations of 53 mg/ml NAD+, 15 mg/ml ketamine, 7 mg/ml lidocaine. Fifth treatment was 3.1 ml of the combination with concentrations of 52 mg/ml NAD+, 16 mg/ml ketamine, 6.5 mg/ml lidocaine. Sixth treatment was 3.1 ml of the combination with concentrations of 52 mg/ml NAD+, 16 mg/ml ketamine, 6.5 mg/ml lidocaine Patient 6 also had noticeable improvement in mood through the course of the treatment by patient's own report and the report of patient's family. Patient 6 expressed at the beginning of the third treatment that during each of Patient's previous weight sessions, following the first two treatments patient had accomplished personal records in the weight room, with the previous evenings bench press being 315 pounds and overhead press being 205 pounds. Given NAD+'s known role in the Krebs citric acid cycle for ATP production, this “side effect” was not terribly surprising. This patient's GAD 7 scores went from 8 to 6 and PHQ 9 scores went from 12 to 10 from pretreatment to 2 weeks after treatment. Patient 6 subjectively reported a total disappearance of his “panic attacks.” Three months later Patient 6 returned for “booster” treatments. First booster dose treatment of 3.3 ml of the combination with concentrations of 48 mg/ml NAD+, 18 mg/ml ketamine, 6 mg/ml lidocaine. Second booster dose was 3.5 ml of the combination with concentrations of 46 mg/ml NAD+, 20 mg/ml ketamine, 6 mg/ml lidocaine.

Patients were sent home with sublingual NAD+ melts with instructions to use these twice a day, which they reported being fairly compliant with.

Of particular interest is the fact that this family spent the time and money to travel back three months after the initial treatment for another series of treatments and brought another daughter with them. They reported that they wished they could have come back at two months instead of three.

Patient 7 was a 27-year-old married mother of 2 who underwent the same essential initial series of the other three patients had undergone 3 months earlier. Treatment began with 2.4 ml of the combination with concentrations of NAD+, 50 mg/ml NAD+, 14.5 mg/ml ketamine, and 4 mg/ml lidocaine. Second dose was 2.4 ml of the combination with concentrations of NAD+, 50 mg/ml NAD+, 14.5 mg/ml ketamine, and 4 mg/ml lidocaine. Third treatment was 2.4 ml of the combination with concentrations of 60 mg/ml NAD+, 20 mg/ml ketamine, and 4 mg/ml lidocaine. Fourth treatment was 2.4 ml of the combination with concentrations of 60 mg/ml NAD+, 20 mg/ml ketamine, and 4 mg/ml lidocaine. Fifth treatment was 2.9 ml of the combination with concentrations of 52 mg/ml NAD+, 21 mg/ml ketamine, and 3 mg/ml lidocaine. The treatments each followed the treatment protocol of rotation to the right, then rotation to the left, then neutral. Some of the rotations were for 5-minute intervals and others were for 15-minute intervals. Patient 7's subjective and family reported mood improvement was quite dramatic. Patient 7's pretreatment GAD 7 score was 11 and her PHQ 9 was 15. 2 weeks after the series Patient 7′s GAD 7 score was 1 and PHQ 9 score was 2. Seven months later Patient 7 returned for booster treatments. First 2.2 ml of the combination with concentrations of 182 mg/ml NAD+, 55 mg/ml ketamine, and 18 mg/ml lidocaine. Second dose 2.2 ml of the combination with concentrations of 182 mg/ml NAD+, 55 mg/ml ketamine, and 18 mg/ml lidocaine. Third dose 2.2 ml of the combination with concentrations of 182 mg/ml NAD+, 55 mg/ml ketamine, and 18 mg/ml lidocaine.

All four of these patient's subjective reports a very positive results over a 10-month period.

### Addiction/Chemical Dependency/Detox

### Example 5

As previously discussed, NAD+ has been utilized in assisting in detox, particularly in assisting patients overcome withdrawal symptoms. Patient 8 was a 25-year-old woman who had flown to Utah from Kansas to go through detox from heroin use. Patient 8 had been smoking between 1-1½ g of heroin per day for several months and had been dealing with substance abuse issues for several years.

Patient 8 was 18-20 hours from a last dose of heroin. Patient 8 was crying and shaking and claiming, “I cannot do this”, despite having been given buprenorphine, Valium and all of the traditional comfort meds for withdrawal. Patient 8 was experiencing severe chemical dependency, specifically withdrawal symptoms. Patient 8 stated “I have to go smoke” referring to heroin. Patient 8 was administered 1 cc of the NAD+/lidocaine mixture to the left nostril. The concentration of the combination was 200 mg/ml NAD+, and 20 mg/ml lidocaine. Patient was crying and restless and it was unclear whether patient would be able to hold the position long enough for absorption. However, 15 minutes later patient was sound asleep. Patient 8 slept for three hours and woke up with no cravings or chemical dependency such as withdrawal symptoms. Patient 8 was treated daily for the next five days. With the same concentrations of the components in the combination, Patient 8 did very well. However, after five days complained of feeling tired and homesick. Patient 8 was asked in more detail about a history of depression and patient stated that “I have tried everything, and nothing works”. The option of adding ketamine to the next treatment was discussed and patient agreed to the trial. Patient 8 had no previous experience with ketamine. This treatment was 1.9 ml with concentrations in the combination of 120 mg/ml NAD+, 18 mg/ml ketamine, and 12 mg/ml lidocaine. The next day patient stated to feeling “wonderful” with mood and emotions much improved. Patient 8 was treated two more times with the combination of NAD+, lidocaine, and ketamine. Patient is doing very well. The second treatment administered 1 ml of the combination with concentrations of 120 mg/ml NAD+, 17.5 mg/ml ketamine and 10 mg/ml lidocaine. The third treatment was 1.8 ml of the combination with concentrations of 130 mg/ml NAD+, 17 mg/ml ketamine, and 13 mg/ml lidocaine.

### Example 6

Patient 9 treatment was at 37-year-old male who has been in and out of detox and recovery many times for many substances over the past 20 years. At the time treatment began, the use of alcohol had been “around the clock” for several months and was about 24 hours from a last drink. Patient 9 stated on admission that “has not felt withdrawal like this before” referring to being more severe than Patient 9 has previously experienced. Patient 9 described being “shaky and tight right now” during initial physical exam and was observed being tremulous and uncomfortable. Patient 9 was sweating. Patient 9 was terrified about having a seizure which Patient 9 stated to having had about a week ago trying to go through chemical dependency, specifically withdrawal without assistance. Patient 9 described a significant reduction in chemical dependency specifically withdrawal symptoms and an immediate “calm” sensation from the NAD+ and lidocaine treatment. Patient 9 also was treated for several days in a row, with ketamine being added during the last couple of days. Patient 9 did not do any follow-up treatment and relapsed about three months later. Patient 9 later returned and described that during the first couple of months there were no cravings despite going to places that would have previously triggered cravings.

Upon return following Patient 9's relapse, Patient 9 stated frankly “I want to do this for six months” which after the initial few days of chemical dependency, specifically withdrawal has continued on a one to two times per week basis for several months with very good results. Patient 9 did have to travel for a couple of weeks for work at one point with no problem. Patient 9 continues to receive treatment at least once a week and usually twice a week. Patient 9 feels that it has made a tremendous difference in recovery and sobriety. Patient 9 states not having the cravings or “hanging on by my fingernails” sensation that has been typical during Patient's sobriety phase in the past. Patient 9 also describes a tremendous reduction in anxiety. Patient has a high stress executive position at work.

Patient 9 was treated with 1 ml of the combination with concentrations of 200 mg/ml of NAD+ and 20 mg/ml lidocaine with neck extension at 30° and rotation 30° to the right and then to the left for 5 minutes each and then neutral for 5 minutes. Patient 9 tolerated the procedure well and noticed a decrease in shakiness and tightness within the first hour. The detox clinic director reported “a difference in attitude, coloring, restfulness, improved sleep, more optimism, and tolerance of residual symptoms”.

Patient 9 received the same treatment for the next 2 days. The fourth treatment increased the dose to 2 ml of the combination at concentrations of 200 mg/ml NAD+ and 20 mg/ml and then was given a 48-hour break. Following the break Patient 9 was then treated daily for three more days. The treatment regime after the break was modified with the addition of ketamine, first with 2.3 ml of the combination at concentrations of 174 mg/ml NAD+, 13 mg/ml and 17 mg/ml lidocaine. Second, with 2.45 ml of the combination at concentrations of 163 mg/ml NAD+, 18 mg/ml ketamine, and 16 mg/ml lidocaine. Third, with 2.8 ml of the combination at concentrations of 143 mg/ml NAD+, 28.5 mg/ml ketamine, and 14 mg/ml lidocaine. Patient 9 reported a complete elimination of cravings and no effect of things or places that would previously have triggered the desire to drink.

Patient 9's GAD 7 scores went from 19 pretreatment to 12 4 months later. His PHQ 9 scores went from 14 to 6.

### Example 7

Patient 10 was a 25-year-old single woman brought in by two other patients who were in treatment with her at their outpatient recovery facility. Patient 10 is a college graduate social worker who has battled severe anxiety and depression since teens and had been on Prozac, buspirone, and Xanax for several years, with a history of other medications being added at various times. Patient 10 had no history of substance abuse but had just gotten out of a very abusive five-year relationship.

Patient 10 was treated with a starting dose of 1.8 ml of the combination with concentrations of 122 mg/ml NAD+, 12 mg/ml lidocaine, and 33 mg/ml ketamine which was well tolerated. Patient 10 came back four days later for a second treatment of 2 ml of the combination with concentrations of 125 mg/ml NAD+, 37.5 mg/ml ketamine, and 12.5 mg/ml lidocaine, and reported to a feeling of “hope for the first time in years”. Patient 10 underwent two more treatments over the next week, one with 2 ml of the combination with concentrations of 125 mg/ml NAD+, 37.5 mg/ml ketamine, and 12.5 mg/ml lidocaine and the other 111 mg/ml NAD+, 44 mg/ml ketamine, and 11 mg/ml, lidocaine and then left to be a bridesmaid at a destination wedding in Hawaii. When Patient 10 came back 10-12 days later patient reported “I went Cliff jumping! I would have never done that before. I just thought why not”. Patient 10 reported that her mother asked her “what has happened to you? You got your pep back.” Patient 10 has continued receiving treatments about once a week for a period of two-and-a-half months and has stopped using all other medications except a half dose of Prozac. Those treatments were 2.5 ml of the combination with concentrations of 100 mg/ml NAD+, 50 mg/ml ketamine, and 10 mg/ml lidocaine.

### Example 8

Patient 11 is a 28-year-old EMT first responder already in treatment following, upon hearing about inventor's treatment. Patient 11 was on buprenorphine for medically assisted detox. Patient 11 did not like the buprenorphine treatment and described a sensation of being constantly in chemical dependency, such as withdrawal while using the buprenorphine. The prior drug of abuse for Patient 11 was heroin. Patient 11 had a ten-year history of addiction with one four-year period of sobriety. Patient 11 has struggled with anxiety and depression for many years. Patient 11 was treated twice a week initially and then once a week during the post-acute withdrawal phase. First treatment was 2.3 ml of the combination with concentration of 130 mg/ml NAD+, 35 mg/ml ketamine, and 13 mg/ml lidocaine. The second treatment was 2.5 ml of the combination with concentration of 120 mg/ml NAD+, 40 mg/ml ketamine, and 12 mg/ml lidocaine. The third was 2.5 ml of the combination with concentration of 120 mg/ml NAD+, 40 mg/ml ketamine, and 12 mg/ml lidocaine. The fourth through tenth treatments were 2.75 ml of the combination with concentration of 109 mg/ml NAD+, 45 mg/ml ketamine, and 10 mg/ml lidocaine. Patient 11 was able to stop using buprenorphine after the first week. Patient 11 expressed a wish to receive treatments more frequently than twice a week initially, however Patient 11 responded well to the treatment. At the eleventh treatment dexamethasone was added to the combination. Patient 11 was administered 3.05 ml of this combination where the concentrations were 98 mg/ml NAD+, 41 mg/ml ketamine, 10 mg/ml lidocaine, and 1 mg/ml. The treatment including dexamethasone was repeated 2 more times. Patient 11's GAD 7 scores were 21 pretreatment and 0 one month later. Patient 12 reported that he has filled out many GAD 7 screening tools over the past several years and has “never put any zeros on them before”. Patient 9 did not fill out a PHQ 9 initially but the posttreatment PHQ 9 was scored at a 3.

### Example 9

Patient 12 is a 46-year-old male who initially did not want to be treated and was first seen when drunk with an estimated, 0.3-0.4 blood alcohol level, based on a level of 0.14 four hours after admission. Patient 12 was very combative. The initial treatment was administered as 1.5 ml of a combination of 133 mg/ml NAD+, 13 mg/ml lidocaine, and 33 mg/ml ketamine. The combination was administered through the left nostril and began with 30-degree rotation to the right side for 10 minutes, then 30-degree rotation to the left side for 10 minutes, followed by neutral position for 10 minutes. Patient 12 responded well to the first treatment. He slept for eight hours and expressed feeling more “solid” where before had felt “fragile”. Confrontational behavior was gone. Prior to this treatment Patient 12 was given Valium and comfort meds every 3-4 hours for the previous 12 hours. For the second treatment of 2.25 ml of a combination of 133 mg/ml NAD+, 33 mg/ml ketamine, and 13 mg/ml lidocaine. The third treatment was 2.25 ml of a combination of 133 mg/ml NAD+, 33 mg/ml ketamine, and 13 mg/ml lidocaine. The fourth treatment was 2.5 ml with concentrations of 120 mg/ml NAD+, 40 mg/ml ketamine, and 12 mg/ml lidocaine. The fifth treatment was 2.75 ml of the combination with 109 mg/ml NAD+, 45 mg/ml ketamine, and 10 mg/ml lidocaine. The sixth treatment was 3 ml of the combination with concentrations of 100 mg/ml NAD+, 50 mg/ml ketamine, and 10 mg/ml lidocaine. The seventh treatment was 3.5 ml of the combination with concentrations of 86 mg/ml NAD+, 57 mg/ml ketamine, and 9.5 mg/ml lidocaine. Treatment went very well for Patient 12. Patient 12's initial GAD score was 17 and the final GAD score was 5.

The invention has been described with reference to various specific and preferred embodiments and techniques. Nevertheless, it is understood that many variations and modifications may be made while remaining within the spirit and scope of the invention.

1. A composition comprising an anesthetic and NAD+.

2. The composition of claim 1, wherein the anesthetic is selected from the group consisting of lidocaine, prilocaine, cocaine, sameridine, bupivacaine, articaine, proparacaine, procaine, and amylocaine.

3. The composition of claim 1, wherein the anesthetic is lidocaine.

4. The composition of claim 1, wherein the composition comprises about 3 mg/ml lidocaine to about 20 mg/ml lidocaine.

5. The composition of claim 1, wherein the composition comprises about 20 mg/ml lidocaine.

6. The composition of claim 1, wherein the composition comprises about 25 mg/ml to about 400 mg/ml NAD+.

7. The composition of claim 1, wherein the composition comprises about 200 mg/ml NAD+.

8. The composition of claim 1, wherein the composition further comprises an NMDA receptor antagonist.

9. The composition of claim 8, wherein the NMDA receptor antagonist is selected from the group consisting of pethidine, levorphanol, ketamine, dextromethorphan (DXM), methoxetamine (MXE), and ketobemidone.

10. The composition of claim 1, wherein the composition comprises ketamine.

11. The composition of claim 1, wherein the composition comprises about 10 mg/ml ketamine to about 200 mg/ml ketamine.

12. The composition of claim 1, wherein the composition comprises about 50 mg/ml ketamine.

13. The composition of claim 1, wherein the composition comprises about 100 mg/ml ketamine.

14. The composition of claim 1, wherein the composition further comprises a corticosteroid.

15. The composition of claim 14, wherein the corticosteroid is selected from the group consisting of cortisone, prednisone, prednisolone, methylprednisolone, dexamethasone, betamethasone, and hydrocortisone.

16. The composition of claim 14, wherein the corticosteroid is dexamethasone.

17. The composition of claim 1, wherein the composition comprises about 0.5 mg/ml dexamethasone to about 3 mg/ml dexamethasone.

18. A method for the treatment of anxiety, depression, or chemical dependency in a subject in need thereof, the method comprising: administering to the subject, the composition of claim 1 to the pterygopalatine fossa of the subject.

19. The method according to claim 18, wherein the subject is administered at least 1ml of the composition.

20. (canceled)

21. (canceled)

22. The method according to claim 18, wherein the chemical dependency is opioid dependency or alcohol dependency.

23. (canceled)

24. (canceled)

25. (canceled)

26. (canceled)
